(19) United States
(12) Patent Application Publication
(10) Pub. No.: US 2018/0153457 A1
(43) Pub. Date: Jun. 7, 2018

(54) DETECTION OF PHYSIOLOGICAL STATE USING THERMAL IMAGE ANALYSIS

(71) Applicant: University of Dayton, Dayton, OH (US)

(72) Inventor: Matthew Montoya, Dayton, OH (US)

(21) Appl. No.: 15/830,433

(22) Filed: Dec. 4, 2017

Related U.S. Application Data
(60) Provisional application No. 62/429,391, filed on Dec. 2, 2016.

(51) Int. Cl. A61B 5/16; G08B 21/18; G06K 9/00; H04N 5/33; A61B 5/00; A61B 5/01

(52) U.S. Cl. CPC A61B 5/164; G08B 21/18; G06K 9/00885; H04N 5/33; G06K 2009/00939; A61B 5/746; A61B 5/015; A61B 5/0008; A61B 5/0013; A61B 5/747

(57) ABSTRACT

Systems, methods, and computer program products for determining a physiological state of an individual. A thermal image of the individual is analyzed to identify thermally responsive and thermally non-responsive regions of the individual. A baseline temperature is determined from the portions of the thermal image covering thermally non-responsive regions, and a response temperature is determined from the portions of the thermal image covering the thermally responsive regions. If the difference between the baseline temperature and the response temperature exceeds a threshold, a determination is made that the individual is having a threat response. This determination may trigger transmission of an alert signal to an emergency responder, who may then locate the individual for further investigation. Thermal images of individuals taken during interrogations may be analyzed in a similar manner to determine if an individual is being deceptive.

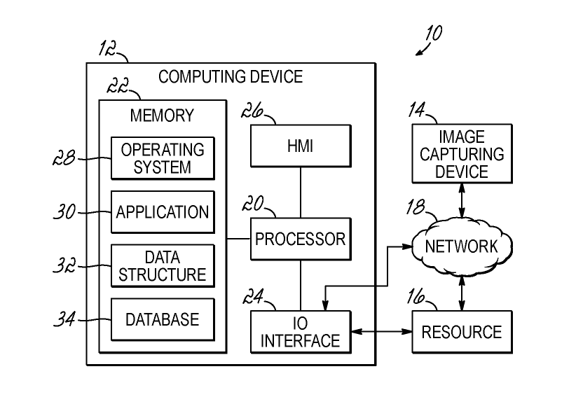

### CROSS-REFERENCE TO RELATED APPLICATIONS

This application claims the benefit of and priority to co-pending U.S. application No. 62/429,391, filed Dec. 2, 2016, the disclosure of which is incorporated by reference herein in its entirety.

### BACKGROUND

This invention relates to imaging technology, and more particularly, to systems, methods, and program products for determining a physiological state of an individual using a thermal image.

Conventional methods of detecting deception, such as polygraphs, measure physiological indicators, such as blood pressure, pulse, respiration, and skin conductivity, while the individual is asked questions. These methods may be applied while the individual is detained, and assume that a guilty individual who is lying will be have a different physiological reaction than an individual who not trying to deceive the interrogator. However, the physiological indicators of an intent to deceive often fail to distinguish between a physiological response to a perceived threat of being caught in an illegal act, and other emotional states, such as confidence that the individual will succeed in overcoming a challenge. Furthermore, such conventional methods of detecting deception also require a significant amount of time to gather the necessary data from the individual, thereby preventing their use in large scale screening.

Imaging systems may be used to monitor individuals and capture certain useful physiological data. Conventional use of imaging systems requires that the imaging system monitor an individual for three-to-five minutes, which is significantly less than methods of detecting deception that use polygraph technology. Imaging systems also typically capture significantly more data in that time span than polygraph testing procedures. Therefore, imaging system based method of detecting deception can be more accurate and efficient than methods of detecting deception that use polygraph technology.

Conventional imaging system based methods use an imaging system, such as an infrared imaging system, to monitor physiological indicators such as changes in heart rate, blood pressure, and electrodermal activity while the individual is detained. These indicators are monitored for changes during a three-to-five-minute time span when the individual is aware that they are being detained for suspected unlawful activity. The guilt of the individual may be ascertained based on whether differences in the physiological indicators represent fear or anxiety in the individual, which could be triggered by a fear of being caught and punished.

However, it is often difficult to determine if the physiological indicators provided by conventional imaging systems are the result of a fear of being caught, or due to some other emotional state or trigger. Such uncertainties can result in significant inaccuracies in determining whether the individual is involved in unlawful activity or is agitated for other reasons. Furthermore, a three-to-five-minute time span prevents conventional imaging based methods from being be applied on a larger scale, such as a part of a security checkpoint or for scanning crowds of people.

Thus, there is a need for improved systems, methods, and computer program products that improve on the ability to detect when an individual is attempting to deceive law enforcement or is engaged in unlawful activity.

### SUMMARY OF THE INVENTION

In an embodiment of the invention, a method of determining a physiological response of an individual is provided. The method includes capturing a thermal image of the individual, identifying a first region of the individual that is thermally non-responsive when the individual has a threat response, and identifying a second region of the individual that is thermally responsive when the individual has the threat response. The method further includes determining a baseline temperature of the first region of the individual, determining a response temperature of the second region of the individual, and determining a difference between the baseline temperature and the response temperature. In response to the difference being greater than a threshold, the method determines that the individual is having a threat response. In response to the difference being less than the threshold, the method determines that the individual is not having the threat response.

In another embodiment of the invention, a system for determining the physiological response of the individual is provided. The system includes an image capturing device, one or more processors in communication with the image capturing device, and a memory in communication with the one or more processors and storing program code. The program code is configures so that, when executed by at least one of the one or more processors, the program code causes the system to capture the thermal image of the individual using the imaging device, identify the first region of the individual that is thermally non-responsive when the individual has the threat response, and identify the second region of the individual that is thermally responsive when the individual has the threat response. The program code further causes the system to determine the baseline temperature of the first region of the individual, determine the response temperature of the second region of the individual, and determine the difference between the baseline temperature and the response temperature. In response to the difference being greater than the threshold, the program code causes the system to determine that the individual is having the threat response. In response to the difference being less than the threshold, the program code causes the system to determine that the individual is not having the threat response.

In another embodiment of the invention, a computer program product is provided. The computer program product includes a non-transitory computer readable storage medium containing program code. The program code is configured to, when executed by one or more processors, cause the one or more processors to capture the thermal image of the individual, identify the first region of the individual that is thermally non-responsive when the individual has the threat response, and identify the second region of the individual that is thermally responsive when the individual has the threat response. The program code is further configured to cause the one or more processors to determine the baseline temperature of the first region of the individual, determine the response temperature of the second region of the individual, and determine the difference between the baseline temperature and the response temperature. In response to the difference being greater than the threshold, the program code causes the one or more processors to determine that the individual is having the threat response. In response to the difference being less than the threshold, the program code causes the one or more processors to determine that the individual is not having the threat response.

The above summary presents a simplified overview of some embodiments of the invention in order to provide a basic understanding of certain aspects the invention described herein. The summary is not intended to provide an extensive overview of the invention, nor is it intended to identify any key or critical elements or delineate the scope of the invention. The sole purpose of the summary is merely to present some concepts in a simplified form as an introduction to the detailed description presented below.

### BRIEF DESCRIPTION OF THE DRAWINGS

The accompanying drawing, which is incorporated in and constitutes a part of this specification, illustrates embodiments of the invention and, together with a general description of the invention given above, and the detailed description given below, serves to explain the invention.

The figure is a diagrammatic view of an exemplary detection system including a computing device and an image capturing device that determine a physiological response of an individual based on a thermal image thereof.

### DETAILED DESCRIPTION

Embodiments of the invention may determine a level of anxiety of an individual based on a physiological response of the individual as the individual performs or contemplates a task or otherwise conducts themselves, such as when passing through a security checkpoint. By quickly and objectively determining the level of anxiety of an individual, embodiments of the invention may allow authorities to identify individuals who are contemplating, in the process of performing, or have recently performed an unlawful activity. These individuals may then be further investigated (e.g., pulled out of line and questioned) before the unlawful activities occur or are completed. Embodiments of the invention may also be used to determine a level of anxiety after an individual has committed an unlawful act, such as while a suspect is trying to escape or is in custody.

Individuals may have different physiological responses when faced with a task that is considered difficult or has an uncertain outcome. One physiological response is a threat response, which tends to increase the level of anxiety in the individual. Another physiological response is a challenge response, which does not tend to increase the level of anxiety in the individual. The individual can also have a passive response, which does not increase the level of anxiety, and may be considered as essentially a non-response. The factors that determine how the individual responds to a task or situation may be based largely on the individual's evaluation of their likelihood of succession. A challenge response or a passive response may result when the individual believes they can accomplish the task or handle the situation. A threat response may result when the individual believes the task is dangerous or difficult, or the individual is otherwise uncertain they can accomplish the task or successfully deal with the situation. In the case of an unlawful task, concerns about being caught may cause the individual to continue having a threat response after the task has been completed.

During a challenge response, the sympathetic nervous system may cause an increase in heart rate, and the adrenal system may release adrenaline. The adrenaline may dilate the individual's blood vessels so that the individual's blood pressure remains relatively constant despite the increase in heart rate. Thus, a challenge response may resemble the individual's response to aerobic activity. A challenge response may be associated with a state of mind of an individual who is not having difficulty engaging a situation, e.g., who is confident they can successfully deal with the situation.

During a threat response, the sympathetic nervous system may cause an increase in heart rate in a similar manner as the challenge response. However, the adrenal system may have a reduced release of adrenaline as compared to the challenge response. This reduced release of adrenaline may result in the blood vessels being constricted as compared to the challenge response, which may cause an increase in blood pressure. A threat response may be associated with a state of mind of an individual who is having difficulty engaging a situation, e.g., due to an excessive level of anxiety from the belief or fear that they will not be able to successfully deal with the situation.

To determine a level of anxiety being experienced by the individual, an imaging device configured to detect the temperature of different regions of the individual, such as an infrared imaging device, may be used to capture a thermal image of the individual. The captured thermal image may be a single thermal image, or one of a series of thermal images comprising a video stream generated as the individual attempts to pass through airport security. The thermal image may be analyzed to identify differences in the temperature between different regions of the individual's skin. The temperatures determined from the thermal image may provide data indicative of the temperature of the skin in regions proximate to various arteries. The temperature of the skin in regions proximate to an artery may be indicative of the temperature of, and thus the amount of blood flowing through, the artery.

It has been determined that there are certain thermally non-responsive regions of an individual which contain arteries that do not constrict when the individual is experiencing a challenge response or a threat response. These regions are also thermally non-responsive when the individual is experiencing a passive response. Because the non-responsive regions are not thermally responsive to either challenges or threats, they may be used to provide a baseline temperature of the individual's skin. In contrast, thermally responsive regions may contain arteries that do not constrict during a challenge response or a passive response, but that do constrict during a threat response. Because the temperature of the thermally responsive regions is affected by threat responses but not challenge responses, they can be used provide a response temperature. The response temperature can be compared to the baseline temperature to distinguish between a threat response and a challenge response in the individual. Thus, a thermal image that shows both thermally responsive and thermally non-responsive regions of the individual may be used determine if an individual's arteries are constricting in response to high levels of anxiety, or due to some other less incriminating emotional state.

Anxiety may be caused by fears about whether the individual can complete the task successfully, fear associated with actual commission of the task (e.g., in the case of a suicide bomber), or a fear of being caught by authorities after completing an unlawful task. Because anxiety typically produces a threat response in the individual, an individual suffering from high levels of anxiety may exhibit significant differences in skin temperature between their thermally responsive and thermally non-responsive regions. Thus, significant differences in skin temperature between thermally responsive and thermally non-responsive regions of the individual may indicate that the arteries of the individual have constricted due to anxiety. Because anxiety may be associated with the commission of unlawful acts, detecting that an individual is exhibiting a threat response may indicate that the individual is contemplating commission of, is committing, or has committed an unlawful act.

In contrast, insignificant differences in temperature between the thermally responsive and thermally non-responsive regions of the individual may indicate that the arteries of the individual have not constricted. This may be due to the individual being relatively free of anxiety (and thus exhibiting a passive response) or confident that they can successfully complete a lawful task, e.g., board a plane and travel to their destination (and thus exhibiting a challenge response). An individual exhibiting a passive response or a challenge response may be presumed as less likely to be associated with criminal activities occurring on or about the time at which the response is detected.

The figure depicts an exemplary detection system 10 for determining an individual's level of anxiety based on a thermal image of the individual. The detection system 10 may include a computing device 12, an image capturing device 14, and an external resource 16. The computing device 12 may communicate with the image capturing device 14 and external resource 16 through a network 18. The network 18 may include one or more private or public data networks (e.g., the Internet) that enable the exchange of data between systems connected to the network 18.

The computing device 12 may include a processor 20, a memory 22, an input/output (I/O) interface 24, and a Human Machine Interface (HMI) 26. The computing device 12 may be operatively coupled to the external resource 16 via the network 18 or I/O interface 24. External resources may include, but are not limited to, servers, databases, mass storage devices, peripheral devices, cloud-based network services, cameras, sensors, or any other resource that may be used by the computing device 12 to implement embodiments of the invention.

The processor 20 may include one or more devices selected from microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on operational instructions that are stored in memory 22. Memory 22 may include a single memory device or a plurality of memory devices including, but not limited to, read-only memory (ROM), random access memory (RAM), volatile memory, non-volatile memory, static random access memory (SRAM), dynamic random access memory (DRAM), flash memory, cache memory, and/or data storage devices such as a hard drive, optical drive, tape drive, volatile or non-volatile solid state device, or any other device capable of storing data.

The processor 20 may operate under the control of an operating system 28 that resides in memory 22. The operating system 28 may manage computer resources so that computer program code embodied as one or more computer software applications, such as an application 30 residing in memory 22, may have instructions executed by the processor 20. The processor 20 may also execute the application 30 directly, in which case the operating system 28 may be omitted. The one or more computer software applications may include a running instance of an application comprising a server, which may accept requests from, and provide replies to, one or more corresponding client applications. One or more data structures 32 may also reside in memory 22, and may be used by the processor 20, operating system 28, and/or application 30 to store or manipulate data.

The I/O interface 24 may provide a machine interface that operatively couples the processor 20 to other devices and systems, such as the external resource 16 or network 18. The application 30 may thereby work cooperatively with the external resource 16 or network 18 by communicating via the I/O interface 24 to provide the various features, functions, applications, processes, and/or modules comprising embodiments of the invention. The application 30 may also have program code that is executed by one or more external resources 16, or otherwise rely on functions or signals provided by other system or network components external to the computing device 12. Indeed, given the nearly endless hardware and software configurations possible, embodiments of the invention may include applications that are located externally to the computing device 12, distributed among multiple computers or other external resources 16, or provided by computing resources (hardware and software) that are provided as a service over the network 18, such as a cloud computing service.

The HMI 26 may be operatively coupled to the processor 20 of computing device 12 to enable a user to interact directly with the computing device 12. The HMI 26 may include video or alphanumeric displays, a touch screen, a speaker, and any other suitable audio and visual indicators capable of providing data to the user. The HMI 26 may also include input devices and controls such as an alphanumeric keyboard, touch screen, a pointing device, keypads, pushbuttons, control knobs, microphones, etc., capable of accepting commands or input from the user and transmitting the entered input to the processor 20.

A database 34 may reside in memory 22, and may be used to collect and organize data used by the various devices, systems, and modules described herein. The database 34 may include data and supporting data structures that store and organize the data. The database 34 may be arranged with any database organization or structure including, but not limited to, a relational database, a hierarchical database, a network database, an object-oriented database, or combinations thereof.

A database management system in the form of a computer software application executing as instructions on the processor 20 may be used to access data stored in records of the database 34 in response to a query, where the query may be dynamically determined and executed by the operating system 28, other applications 30, or one or more modules. Although embodiments of the invention may be described herein using relational, hierarchical, network, object-oriented, or other database terminology in specific instances, embodiments of the invention may use any suitable database management model, and are not limited to any particular type of database.

Image capturing device 14 may comprise a thermal imaging camera that includes one or more image sensors which capture temperature data in the form of a thermal image. The image capturing device may also include an optical imaging camera or capability, and may be configured to capture optical images of all or a portion of the area that is being thermally imaged. The thermal image may be generated base on long-wavelength infrared radiation received at the image capturing device 14. Long-wavelength infrared radiation may include electromagnetic radiation having wave-lengths that cover the peak emission wavelength of objects having temperatures in the range of normal human body temperatures. For example, electromagnetic radiation having wavelengths between 9,000 and 14,000 nanometers.

Temperature data may include data that depicts an absolute temperature or relative temperature between different regions of an image including an individual. Image capturing device 14 may be configured to detect the absolute temperature of different regions of the individual, and/or the relative temperature between different regions of the individual. Image capturing device 14 may capture the thermal image of the individual by taking a single image, or may capture a series of images and/or generate video feed over a period of time. More specifically, image capturing device 14 may be configured to detect the temperature of different arterial regions of the individual in a single image of the individual.

The image capturing device 14 may transmit the thermal image, optical image, or captured temperature data to the computing device 12 over the network 18 as one or more data packets. Each thermal image may indicate the temperature of points in the image using different levels of luminance and/or different colors, e.g., with brighter and/or redder regions indicating relatively high temperatures, and dimmer and/or bluer regions indicating relatively cooler temperatures. The computing device 12 may display thermal images, optical images, or temperature data via the HMI 26.

The detection system 10 may process the thermal image or temperature data to determine the level of blood flow in different regions of the individual. The detection system 10 may then determine a level of anxiety of the individual based on the determined blood flows. Rather than merely gathering physiological data that could be indicative of several different emotional or physical states of an individual, the thermal data associated with the level of blood flow of the individual may be directly correlated with the level of anxiety being experienced by the individual. Other physiological indicators, such as an increase in heart rate, may result due to reasons other than an increase in the individual's level of anxiety. For example, the heart rate of the individual may be increased due to the individual rushing to catch a flight for which they are running late. Thus, although the individual may not be anxious about being identified as a security risk when passing through security, they would nevertheless have an increased heart rate from the physical exertion of rushing to catch their flight.

As the individual performs a task, the temperature of the individual may fluctuate based on whether the individual is anxious about failing to complete the task, or is confident they will successfully complete the task. An increase in the level of anxiety experienced by an individual may trigger constriction of certain arteries. Constriction in the arteries may in turn cause a decrease in the volume of blood flowing through those arteries. A decrease in the volume of blood flowing through those arteries may further result in a decrease in the temperature of the skin in regions proximate to those arteries.

In contrast, when the individual is confident they will successfully complete a task, their arteries may experience little of no change in constriction levels, or may even dilate. Dilation of the arteries may result in an increase in the volume of blood flowing through those arteries, which may cause in an increase in the temperature of regions proximate to the arteries. In cases where the arteries do not experience an increase in constriction, the temperature of regions proximate to those arteries may remain constant. Likewise, a lack of anxiety may result in no change in the arteries such that no substantial change in blood flow occurs, resulting in no substantial change in temperature.

Advantageously, by comparing changes in the temperature of different regions of an individual as indicated by a thermal image, the detection system 10 may be able to distinguish individuals experiencing a threat response from individuals experiencing a challenge response or a passive response. A determination that the individual is experiencing a threat response may result in a psychological assessment that the individual is having difficulty dealing with their current situation. In contrast, determination that the individual is experiencing a challenge response may result in a psychological assessment that the individual believes they can effectively deal with their current situation. Thus, the ability to distinguish threat responses from challenge responses (and passive responses) may increase the effectiveness of the detection system 10 as compared to conventional thermal imaging based deception detecting systems.

Detecting deception and/or individuals contemplating unlawful acts based on a single image of the individual may also improve the response time of the system. Due to the extended nature of an individual's physiological response to contemplating, committing, and leaving the scene of a crime, physiological response determinations made before, during, and after the individual commits the crime may be all useful in identifying the individual as a suspect.

By way of example, an individual attempting to smuggle drugs onto the country may entertain thoughts of whether they can successfully smuggle the contraband past airport security, and what will happen to them if they are not successful. How the individual evaluates the answer to those questions may result in a threat response if the individual is afraid they will not be able to successfully pass through security. If the individual is not carrying contraband and has no reason to fear passing through security, the individual is more likely to have a passive response or a challenge response. Thus, the individual's own perception as to whether they can successfully complete a task may be sufficient to generate physiological response in accordance with the nature of the task.

The circulatory system transfers heat throughout the body, and blood flow is largely responsible for the temperature of certain regions of the body. Changes in the heat transferred by blood flow in large vessels (e.g., radial, femoral, and/or carotid arteries) and in capillaries due to a threat response may be minimal. Thus, exposed regions of the individual near these types of arteries may experience minimal changes in temperature when the individual is experiencing either a threat response or a challenge response. In contrast, changes in the heat transferred by blood flow through arteries supplying muscle tissue and through terminal arteries due to a threat response can result in significant changes in the temperature of those regions as compared to a challenge response.

Thus, whether an individual is having a threat response or some other response may be determined by comparing the temperature of thermally responsive regions of the individual, which are associated with arteries that experience significant changes in blood flow due to the threat response, with the temperature of thermally non-responsive regions of the individual associated with arteries that experience minimal changes in blood flow due to the threat response. The difference in temperature between the thermally responsive regions of the individual and the thermally non-responsive regions of the individual may be correlated with a likelihood that the individual is having a threat response. Similar differences in temperature may occur before, during, and after the individual performs the task causing the threat response.

Thermally responsive regions of the individual may include extremities, such as the hands and fingers, and may have a significant difference in temperature when the individual is having a threat response. The arteries in the hand regions and finger regions may constrict significantly in response to the individual experiencing anxiety or fear. This constriction may reduce the amount of blood circulating in the hands and fingers sufficiently to produce a significant decrease in the temperature of these regions of the individual. The blood that is no longer circulating in the hands and fingers may be redirected to the larger arteries of the neck region and the core region of the individual. Because these larger arteries carry a significant volume of blood flow even when the arteries in the hands and fingers are not constricted, the increase in blood flow due to the arteries in the hands and fingers being constricted may not have a significant impact on the temperature of the neck and/or core regions.

The decrease in the temperature of the hands and fingers triggered by the threat response may be more pronounced in the fingers, and most particularly in the distal fingers that are furthest from the heart. Thus, a thermal image showing a significant temperature difference between the hands and/or fingers and the neck and/or core (e.g., a temperature difference that exceeds a predetermined threshold) may indicate that the individual is having a threat response. In general, regions more distal from the heart may cool more dramatically during a threat response as compared to regions more proximate to the heart. For example, the lower forearm region may also experience significant changes in temperature when the individual has a threat response.

In an individual having a passive or challenge response, the arteries in the hands and fingers may be unconstricted and/or dilated. This may cause a normal or increased volume of blood to circulate through the hands and fingers so that the temperature of these regions is maintained or increased as compared to the baseline temperature. The temperature of the neck and/or core regions may remain unchanged since any changes in blood flow through the hands and fingers due to the challenge response will typically not significantly alter the volume of blood flowing in the arteries of the neck and/or core regions. An image captured by image capturing device 14 that shows an insignificant temperature difference between the hand and/or finger regions verses the neck and/or core regions (e.g., a temperature difference that is below the predetermined threshold) may be indicative that the individual is having passive or challenge response.

Three facial arteries may be associated with responsive and non-responsive regions of the individual when the individual has a threat response. These arteries include the ophthalmic artery, which delivers blood to the forehead, the angular artery, which delivers blood to the nose, and the maxillary arteries, which deliver blood to the cheeks and upper nasal regions. The regions of the individual that receive blood from these arteries include five discrete facial regions. These regions may be analyzed to determine the type of physiological response the individual is having, and include the tip of the nose, the cheeks, the neck, the forehead, and the chin. The nose may cool during a threat response due to constriction of the angular artery. In contrast, the temperature of neck and outer forehead regions may remain relatively constant during a threat response. Thus, comparing the temperature of the nose to the temperatures of the neck and forehead may allow the detection system to determine the type of physiological response an individual depicted in a thermal image is having.

Thermal images captured by the image capturing device 14 may typically include one or more of the face, neck, arms, and hands. Regions that are generally not thermally responsive to a threat response which may be used to determine a baseline temperature for comparison may include the neck, upper forearm, forehead, and chin. Regions that are thermally responsive to a threat response may include the nose and the fingers, particularly the tip of the nose and distal fingers. Regions that may experience increase blood flow during a threat response may include the cheeks and upper lip. The temperature of regions experiencing vasodilatation may increase during a threat response due to an increased amount of blood flow. Thus, these regions may experience an upward temperature change from their baseline temperature.

Typically, the most significant changes in temperature may be cooling that results from constriction of the arteries as described above. Due to the larger temperature changes experience by responsive regions from vasoconstriction, these regions may enable a more reliable determination that the individual is having a threat response than increases in temperature due to vasodilation or blood flow diverted from vasoconstricted regions.

Due to the different responses of certain regions of an individual discussed above, analyzing a thermal image of an individual may allow the detection system 10 to determine a level of anxiety in the individual. This analysis may be based on temperature differences between regions of the human body proximate to large vessels or small capillaries, and regions of the human body proximate to arteries supplying muscle tissue or terminal arteries. The amount of temperature difference detected may be correlated to the amount of constriction or dilation of the arteries, which in turn may be associated with the physiological response of the individual.

In operation, the computing device 12 may receive one or more thermal images of individuals passing through an area, such as an airport terminal, from the image capturing device 14. The thermal images may include one or more individuals. An image analysis application 30 running on the computing device 12 may analyze the thermal profile of each individual in each image, and determine a temperature difference between the thermally responsive regions of the individual and the thermally non-responsive regions of the individual. In response to this temperature difference exceeding a threshold, the computing system 12 may generate an alert signal. The alert signal may be provided by the HMI 26 of computing device 12 to notify nearby personnel, or may be transmitted (e.g., as an electronic message) from the computing device 12 to another computing device connected to the network 18, such as a smartphone carried by a security officer or a computing system operated by another emergency responder, e.g., the Department of Homeland Security, airport security, Emergency 911, etc. The alert signal may cause the HMI 26 to emit audio and/or visual stimuli that indicate to someone near the alerting device that the detection system 10 has detected an individual exhibiting a threat response. The alert signal may also provide information such as an optical or thermal image of the individual, or a location of the individual, to help the emergency responder find the individual in question. Once the individual has been found, security personnel may observe the individual and/or intervene, e.g., by detaining the individual, to determine whether the individual is involved in criminal activity.

In another embodiment of the invention, a police officer may obtain one or more thermal images of an individual while the individual is being followed or detained. The thermal images may be obtained using a portable image capturing device, an image capturing device installed in a detention area or on a patrol vehicle, or any other device that can capture thermal images and transmit the thermal images to the computing device 12, or perform an analysis of the thermal images locally. The thermal images may be captured surreptitiously, or with the knowledge of the individual. In response to receiving a thermal image from the imaging device, the computing device 12 may analyze the image and provide a report to the police officer. The report may include a conclusion or a probability that the individual is having a threat response, challenge response, passive response, or some other physiological response, based on an analysis of the thermal image. The type of physiological response the individual is exhibiting while detained may help the police officer determine if the individual should be further investigated (e.g., taken into custody for further questioning) or released.

The ability to distinguish individuals that are frightened or anxious from those that are merely agitated or confident may improve security screening and/or monitoring in many situations. Exemplary applications of the detection system 10 may include, but are not limited to, border and/or airport security checkpoints (e.g., to identify individuals who may be attempting to smuggle contraband across a boarder or onto a plane), police interrogations (e.g., to allow police officers to determine when an individual is being deceptive), field and combat threat assessment (e.g., by enabling soldiers to rapidly detect which individuals in a crowd pose the greatest risk), vigilance research (e.g., allowing supervisors to determine when an operator of a machine is losing focus or can no longer safely operate the machine), workload assessment (e.g., by detecting when workers are experiencing excessive cognitive workload), and gaming consoles (e.g., by allowing the game to adjust play difficulty based on the player's physiological response).

In general, the routines executed to implement the embodiments of the invention, whether implemented as part of an operating system or a specific application, component, program, object, module or sequence of instructions, or a subset thereof, may be referred to herein as “computer program code,” or simply “program code”. Program code typically comprises computer-readable instructions that are resident at various times in various memory and storage devices in a computer and that, when read and executed by one or more processors in a computer, cause that computer to perform the operations necessary to execute operations and/or elements embodying the various aspects of the embodiments of the invention. Computer-readable program instructions for carrying out operations of the embodiments of the invention may be, for example, assembly language or either source code or object code written in any combination of one or more programming languages.

Various program code described herein may be identified based upon the application within which it is implemented in specific embodiments of the invention. However, it should be appreciated that any particular program nomenclature which follows is used merely for convenience, and thus the invention should not be limited to use solely in any specific application identified and/or implied by such nomenclature. Furthermore, given the generally endless number of manners in which computer programs may be organized into routines, procedures, methods, modules, objects, and the like, as well as the various manners in which program functionality may be allocated among various software layers that are resident within a typical computer (e.g., operating systems, libraries, API's, applications, applets, web based services, etc.), it should be appreciated that the embodiments of the invention are not limited to the specific organization and allocation of program functionality described herein.

The program code embodied in any of the applications/modules described herein is capable of being individually or collectively distributed as a program product in a variety of different forms. In particular, the program code may be distributed using a computer-readable storage medium having computer-readable program instructions thereon for causing a processor to carry out aspects of the embodiments of the invention.

Computer-readable storage media, which is inherently non-transitory, may include volatile and non-volatile, and removable and non-removable tangible media implemented in any method or technology for storage of information, such as computer-readable instructions, data structures, program modules, or other data. Computer-readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. A computer-readable storage medium should not be construed as transitory signals per se (e.g., radio waves or other propagating electromagnetic waves, electromagnetic waves propagating through a transmission media such as a waveguide, or electrical signals transmitted through a wire). Computer-readable program instructions may be downloaded to a computer, another type of programmable data processing apparatus, or another device from a computer-readable storage medium or to an external computer or external storage device via a network.

Computer-readable program instructions stored in a computer-readable medium may be used to direct a computer, other types of programmable data processing apparatuses, or other devices to function in a particular manner, such that the instructions stored in the computer-readable medium produce an article of manufacture including instructions that implement the functions, acts, and/or operations specified in the flow-charts, sequence diagrams, and/or block diagrams. The computer program instructions may be provided to one or more processors of a general-purpose computer, a special purpose computer, or other programmable data processing apparatus to produce a machine, such that the instructions, which execute via the one or more processors, cause a series of computations to be performed to implement the functions, acts, and/or operations specified in the flow-charts, sequence diagrams, and/or block diagrams.

In certain alternative embodiments, the functions, acts, and/or operations specified in the flow-charts, sequence diagrams, and/or block diagrams may be re-ordered, processed serially, and/or processed concurrently consistent with embodiments of the invention. Moreover, any of the flow-charts, sequence diagrams, and/or block diagrams may include more or fewer blocks than those illustrated consistent with embodiments of the invention.

The terminology used herein is for the purpose of describing particular embodiments only and is not intended to be limiting of the embodiments of the invention. As used herein, the singular forms “a”, “an” and “the” are intended to include the plural forms as well, unless the context clearly indicates otherwise. It will be further understood that the terms “comprises” and “comprising,” when used in this specification, specify the presence of stated features, integers, actions, steps, operations, elements, or components, but do not preclude the presence or addition of one or more other features, integers, actions, steps, operations, elements, components, or groups thereof. Furthermore, to the extent that the terms “includes”, “having”, “has”, “with”, “comprised of”, or variants thereof are used in either the detailed description or the claims, such terms are intended to be inclusive in a manner similar to the term “comprising”.

While all of the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. A method of determining a physiological response of an individual, the method comprising:
capturing a thermal image of the individual;
identifying a first region of the individual that is thermally non-responsive when the individual has a threat response;
identifying a second region of the individual that is thermally responsive when the individual has the threat response;
determining a baseline temperature of the first region of the individual;
determining a response temperature of the second region of the individual;
determining a difference between the baseline temperature and the response temperature;
in response to the difference being greater than a threshold, determining that the individual is having a threat response; and
in response to the difference being less than the threshold, determining that the individual is not having the threat response.

2. The method of claim 1 further comprising:
in response to determining the individual is having the threat response, generating an alert signal.

3. The method of claim 2, wherein generating the alert signal comprises:
generating an electronic message that identifies a location of the individual; and
transmitting the electronic message to an emergency responder.

4. The method of claim 2, wherein generating the alert signal comprises:
generating an electronic message that includes at least one of an optical image of the individual or the thermal image of the individual; and
transmitting the electronic message to an emergency responder.

5. The method of claim 1, wherein the second region is one of a plurality of second regions, and determining the difference between the baseline temperature and the response temperature comprises:
determining the difference between the baseline temperature and the response temperature of each of the second regions to generate a plurality of differences; and
in response to the difference being greater than a respective threshold for at least one difference of the plurality of differences, determining that the individual is having the threat response.

6. The method of claim 5 wherein the respective threshold for each difference of the plurality of differences is set independently of the other thresholds.

7. The method of claim 1, wherein the first region is selected from a chin, a neck, or an upper forearm region of the individual, and the second region is selected from a nose or a finger region of the individual.

8. The method of claim 1, wherein determining that the individual is not having the threat response includes determining that the individual is having a challenge response or a passive response.

9. The method of claim 1 wherein the thermal image is generated using long-wavelength infrared radiation.

10. The method of claim 1 further comprising:
capturing an optical image of the individual.

11. The method of claim 1 wherein the thermal image is one of a series of thermal images comprising a video stream.

12. A system for determining a physiological response of an individual, the system comprising:
an image capturing device;
one or more processors in communication with the image capturing device; and
a memory in communication with the one or more processors and storing program code that, when executed by at least one of the one or more processors, causes the system to:
capture a thermal image of the individual using the imaging device;
identify a first region of the individual that is thermally non-responsive when the individual has a threat response;
identify a second region of the individual that is thermally responsive when the individual has the threat response;
determine a baseline temperature of the first region of the individual;
determine a response temperature of the second region of the individual;
determine a difference between the baseline temperature and the response temperature;
in response to the difference being greater than a threshold, determine that the individual is having a threat response; and
in response to the difference being less than the threshold, determine that the individual is not having the threat response.

13. The system of claim 12 wherein the program code is further configured to cause the system to:
in response to determining the individual is having the threat response, generate an alert signal.

14. The system of claim 13, wherein the program code generates the alert signal by causing the system to:
generate an electronic message that identifies a location of the individual; and
transmit the electronic message to an emergency responder.

15. The system of claim 13, wherein the program code generates the alert signal by causing the system to:
generate an electronic message that includes at least one of an optical image of the individual or the thermal image of the individual; and
transmit the electronic message to an emergency responder.

16. The system of claim 12, wherein the second region is one of a plurality of second regions, and the program code determines the difference between the baseline temperature and the response temperature by causing the system to:
determine the difference between the baseline temperature and the response temperature of each of the second regions to generate a plurality of differences; and
in response to the difference being greater than a respective threshold for at least one difference of the plurality of differences, determine that the individual is having the threat response.

17. The system of claim 16 wherein the respective threshold for each difference of the plurality of differences is set independently of the other thresholds.

18. The system of claim 12, wherein the first region is selected from a chin, a neck, or an upper forearm region of the individual, and the second region is selected from a nose or a finger region of the individual.

19. The system of claim 12 wherein the thermal image is generated using long-wavelength infrared radiation.

20. A computer program product for determining a physiological response of an individual, the computer program product comprising:
a non-transitory computer readable storage medium containing program code that, when executed by one or more processors, causes the one or more processors to:
capture a thermal image of the individual;
identify a first region of the individual that is thermally non-responsive when the individual has a threat response;
identify a second region of the individual that is thermally responsive when the individual has the threat response;
determine a baseline temperature of the first region of the individual;
determine a response temperature of the second region of the individual;
determine a difference between the baseline temperature and the response temperature;
in response to the difference being greater than a threshold, determine that the individual is having a threat response; and
in response to the difference being less than the threshold, determine that the individual is not having the threat response.
